(19) United States
(12) Patent Application Publication
(10) Pub. No.: US 2025/0072956 A1
(43) Pub. Date: Mar. 6, 2025

(54) ELECTRODE FOR AN ELECTROSURGICAL HANDHELD INSTRUMENT

(71) Applicant: OLYMPUS WINTER & IBE GMBH, Hamburg (DE)

(72) Inventor: Harald Hanke, Hamburg (DE)

(73) Assignee: OLYMPUS WINTER & IBE GMBH, Hamburg (DE)

(21) Appl. No.: 18/742,467

(22) Filed: Jun. 13, 2024

Related U.S. Application Data
(60) Provisional application No. 63/535,101, filed on Aug. 29, 2023.

(51) Int. Cl. A61B 18/14; A61B 18/00

(52) U.S. Cl. CPC A61B 18/149; A61B 2018/00601; A61B 2018/0091; A61B 2018/144

(57) ABSTRACT

An electrosurgical treatment can be carried out with an electrode reliably and safely even if metallic elements are present in the region to be treated. For this purpose, an electrode for an electrosurgical handheld instrument is made of an electrically conductive wire, the two ends of the wire being connectable to an electrode carrier of the handheld instrument. The wire has two portions R and L, which are adjacent to the two ends of the electrode carrier and can be aligned parallel to one another and rectilinearly. Next to these two portions R and L, there is a portion C that connects the two portions R and L to one another. The wire has a cross section of from 0.5 mm to 1.0 mm.

The invention relates to an electrode for an electrosurgical handheld instrument according to the preamble of claim 1.

Electrosurgical handheld devices, in particular resectoscopes, of the type in question are used especially for electrosurgical operations in urology. In this case, these devices are conventionally used for the resection and evaporation of tissue, for example tissue in the lower urinary tract. For this purpose the handheld device, in particular the resectoscope, may have a longitudinally displaceable electrode carrier which, after the insertion of the device with a distal working end into the body to be treated, can be displaced forward from a distal end of the instrument shaft of the handheld device. An electrosurgical electrode is arranged at a distal end on the electrode carrier. This electrode may for example have the shape of a loop, and in order to manipulate the tissue is pulled or pushed through the tissue, depending on the design of the instrument.

Known electrodes are formed from a wire. For loop electrodes, for example, it is known that the wire is formed into a loop and the two free ends are connected to the electrode carrier. By the application of a radiofrequency alternating voltage, a plasma is ignited on the electrode so that a plasma is formed on the wire in interaction with the fluid surrounding the electrode. The plasma is preferentially ignited at positions of the electrode which have a small radius of curvature, since the electric field strength is greatest there.

In radiofrequency surgery, situations in which the patient has been fitted in previous treatments with metallic implants or other elements consisting of an electrically conductive material in the region of the tissue to be treated have been found to be problematic. When a radiofrequency electrical potential is applied to the electrode, on the one hand a discharge or sparking takes place between the electrode and the metallic element. This leads to uncontrolled and even unintended manipulation of the human tissue. The resulting traumatization of the patient is unpredictable, and should therefore be avoided. On the other hand, the electrode experiences a greatly increased level of erosion due to this discharge, so that the risk of breaking is exacerbated. Furthermore, the electrode may be directly melted by a very high current strength occurring briefly and in a locally limited fashion. The two break points of the electrode, or the two free ends, may cause serious injury to the patient. Furthermore, there is the risk that an arc will be formed between the two ends of the break, which again leads to uncontrolled manipulation of the tissue.

The object of the invention is therefore to provide an electrode with which an electrosurgical treatment can be carried out reliably and safely even if metallic elements are present in the region to be treated.

A solution to this object is described by claim 1. Accordingly, an electrode for an electrosurgical handheld instrument, in particular for a resectoscope, consists of an electrically conductive wire, the two ends of the wire being connectable to an electrode carrier of the handheld instrument. The wire has two portions R and L, which are adjacent to the two ends of the electrode carrier and can be aligned parallel to one another and rectilinearly. Next to these two portions R and L, there is a portion C that connects the two portions R and L to one another. According to the invention, the wire has a cross section of from 0.5 mm to 1.0 mm. The effect of this significantly increased cross section of the electrode wire is that the electrode has a sufficient strength even in the event of an unforeseeable flashover and does not break, at least during the activated time. Because of the greater mass in comparison with known electrodes, a material loss due to erosion is less serious. According to one particularly preferred exemplary embodiment of the invention, the wire may have a cross section of 0.8 mm.

Preferably, the two portions R and L of the wire may be aligned parallel to one another. Likewise, however, it is also conceivable for the two portions R and L to include an angle and/or have a radius. Likewise, it is conceivable for the portion C to be configured as an arc or bow. Depending on the type of treatment and embodiment of the electrode, it may have almost any desired shape. For example, it is also conceivable for the portions R, L and C to consist of further sub-portions which include an angle between them or a radius. Owing to this flexibility in the shape of the electrode, it is conceivable to use a specially configured electrode for every application.

According to a further particularly advantageous exemplary embodiment, the wire consists of an electrically conductive material with a very high melting point, for example tungsten, molybdenum or tantalum. It has been found that these materials are particularly suitable for the application described above. The melting point should be at least 2000° C., preferably more than 3000° C., in particular more than 3400° C.

Further, according to the invention, the wire may have a round or circular cross section. This choice of the shape of the electrode allows particularly economical production. Ultimately, a wire of the material mentioned above needs to be bent into the correct shape in order to produce the electrode. This production on the one hand can be carried out without great outlay and above all reproducibly, so that the required economical production is ensured, particularly for so-called single-use tools.

Preferentially, the portions R, L and C may lie in a plane or the portions R, L and C may lie in two different planes. According to a further exemplary embodiment, the portions R, L and C may be arranged at a right angle to one another.

A preferred exemplary embodiment of the invention will be described in more detail below with the aid of the drawing, in which:

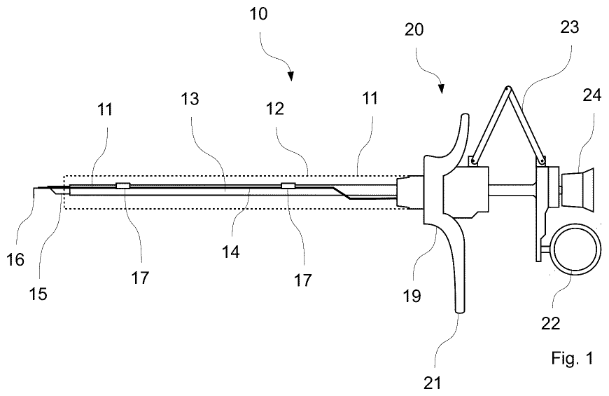

FIG. 1 shows a schematic representation of a surgical handheld device, in particular a resectoscope,

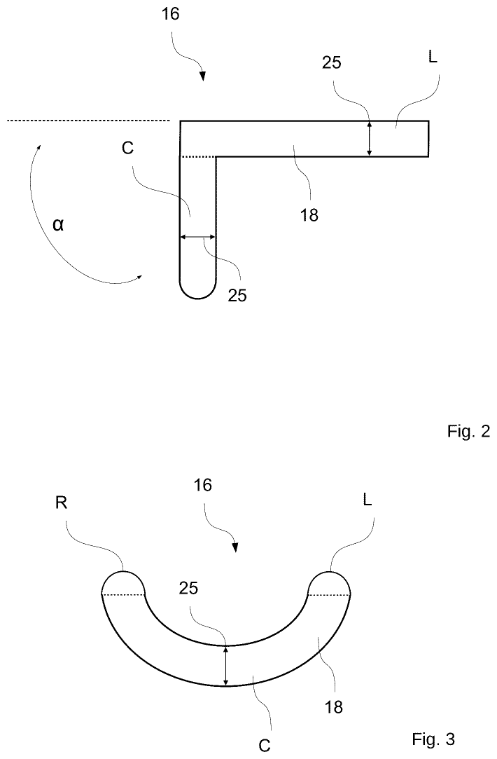

FIG. 2 shows a lateral view of an electrode, and

FIG. 3 shows a front view of the electrode.

FIG. 1 shows a schematic lateral sectional representation of a known resectoscope 10. The resectoscope 10 has a resectoscope shaft 11, which comprises an outer shaft 12, or a cover tube. A tubular inner shaft 13 runs inside the outer shaft 12. Represented inside the inner shaft 13, there are an electrode carrier 14 and optics 15 (merely indicated). Other elements (not represented here) may furthermore be arranged in the resectoscope 10, for example a separate rinsing tube and the like.

At a distal end, the electrode carrier 14 has an electrosurgical tool or an electrode 16. The electrode 16 represented here is depicted as a loop, although it may also have another shape.

By actuating a handle 19, the electrode carrier 14 can be moved while being forcibly guided axially in the distal and proximal directions. It may in this case be deployed beyond the distal end of the inner shaft 13 and the outer shaft 12. This allows the operator to manipulate tissue even further away from the resectoscope tip. For this purpose, the inner shaft 13 and/or the electrode carrier 14 may be mounted rotatably about their longitudinal axis. For the manipulation of the tissue, a radiofrequency electrical current is applied to the electrode 16.

The resectoscope 10 represented in FIG. 1 has a passive transporter, in which a slide 20 is displaced by relative movement of the grip parts 21 and 22 arranged proximally on the resectoscope shaft 11 against a spring force applied by a spring bridge 23 in the distal direction against the distal first grip part 21. During the displacement of the slide 20 in the distal direction against the grip part 21, the electrode carrier 14 is displaced (in a manner not represented) in the distal direction. When the grip parts 21, 22 are released, the spring force generated by the spring bridge 23 forces the slide 20 back into its initial position, the electrode carrier 14 being pulled in the proximal direction. During the return displacement of the slide 20, an electrosurgical intervention with the electrode 16 may be carried out without manual force from the operator, that is to say passively.

For expedient treatment by means of the electrode 16, the optics 15 are positioned in such a way that the operator is provided with an optimal view of the region of the operation. For this purpose, the resectoscope 10 has, at a proximal end, an eyepiece 24 which is connected to the optics 15. Alternatively, it is also conceivable for a camera to be arranged on the resectoscope 10 instead of the eyepiece 24.

FIGS. 2 and 3 represent an exemplary embodiment of an electrode 16 according to the invention. This electrode 16 has portions R and L, these portions R and L being of equal length and aligned parallel to one another. The portions R and L are connected to the distal ends of the electrode carrier tubes.

The central portion C is located between the portions R and L. In the exemplary embodiment of the electrode 16 as represented here, the portion C, or the plane in which the portion C is located, is rotated by an angle α=90° in relation to a plane defined by the two portions R and L. It is likewise conceivable for the angle α to have another value.

The length of the two portions R and L may be from a few millimeters to 2 cm, a distance between the two portions R and L being a few millimeters. According to the invention described here, the cross section 25 of the electrode 16 on all portions R, L and C is from 0.5 mm or 1.0 mm, preferably from 0.7 mm to 0.9 mm, in particular 0.8 mm. It has been found that such dimensioning of the cross section 25 behaves advantageously especially when there are further metallic objects in the vicinity of the region to be treated.

Besides the loop shape of the electrode 16 as represented here by way of example, it is furthermore conceivable for the region C to have another shape, for example a loop with a smaller or greater radius of curvature. Likewise, it is conceivable for the portion C to consist of a plurality of sub-portions, which respectively include an angle.

### LIST OF REFERENCE SIGNS

10 resectoscope
11 resectoscope shaft
12 outer shaft
13 inner shaft
14 electrode carrier
15 optics
16 electrode
17 guide element
18 wire
19 handle
20 slide
21 grip part
22 grip part
23 spring bridge
24 eyepiece
25 cross section
R portion
L portion
C portion
α angle

1-10. (canceled)

11. An electrode for an electrosurgical handheld instrument consisting of an electrically conductive wire, the two ends of the wire being connectable to an electrode carrier of the handheld instrument, wherein the wire has a cross section of from 0.5 mm to 1.0 mm.

12. The electrode for an electrosurgical handheld instrument as claimed in claim 11, wherein the wire has two portions R and L, the two portions R and L adjoining the electrode carrier with two first ends and there being a portion C that connects the two portions R and L to one another between two ends of the portions R and L.

13. The electrode for an electrosurgical handheld instrument as claimed in claim 11, wherein the two portions R and L of the wire are aligned parallel to one another and rectilinearly.

14. The electrode for an electrosurgical handheld instrument as claimed in claim 11, wherein the portion C is configured as an arc or bow.

15. The electrode for an electrosurgical handheld instrument as claimed in claim 11, wherein the wire consists of an electrically conductive material with a very high melting point.

16. The electrode for an electrosurgical handheld instrument as claimed in claim 15, wherein the melting point is at least 2000° C.

17. The electrode for an electrosurgical handheld instrument as claimed in claim 11, wherein the wire has a round or circular cross section.

18. The electrode for an electrosurgical handheld instrument as claimed in claim 11, wherein the portions R, L and C lie in a plane, or wherein the portions R, L and C lie in two different planes.

19. The electrode for an electrosurgical handheld instrument as claimed in claim 11, wherein the portions R, L and C are arranged at a right angle to one another.

20. The electrode for an electrosurgical handheld instrument as claimed in claim 11, wherein the wire has a cross section of 0.8 mm.
